(19) United States
(12) Patent Application Publication
(10) Pub. No.: US 2010/0034902 A1
(43) Pub. Date: Feb. 11, 2010

(54) METHOD FOR THE DIAGNOSIS AND TREATMENT OF SALT SENSITIVITY AND RELATED CONDITIONS

(75) Inventors: Maria Carolina Delgado, Doral, FL (US); Bertram Pitt, Ann Arbor, MI (US)

Correspondence Address: Casimir Jones, S.C., 2275 DEMING WAY, SUITE 310, MIDDLETON, WI, 53562, US

(73) Assignee: THE REGENTS OF THE UNIVERSITY OF MICHIGAN, Ann Arbor, MI (US)

(21) Appl. No.: 12/522,248

(22) PCT Filed: Jan. 17, 2008
(86) PCT No.: PCT/US08/51320

§ 371 Date: Jul. 6, 2009

Related U.S. Application Data
(60) Provisional application No. 60/881,415, filed on Jan. 19, 2007.

(51) Int. Cl. A61K 33/04; A61K 33/00; C12Q 1/02; A61K 31/585; A61K 31/546; A61K 31/4406; A61K 31/4155; A61K 31/41; A61K 31/4178; A61K 31/4168; A61K 31/403; A61K 31/40; A61K 31/355; A61K 31/197; A61K 31/135; A61K 31/138; A61K 31/04

(52) U.S. Cl. 424/702; 424/722; 435/29; 514/173; 514/222.5; 514/355; 514/381; 514/382; 514/397; 514/401; 514/411; 514/412; 514/423; 514/458; 514/567; 514/651; 514/652; 514/742

(57) ABSTRACT

The present invention provides methods for measuring, detecting, diagnosing, treating, and researching salt sensitivity and related conditions. In particular, the present invention provides methods for measuring, detecting, diagnosing, treating, and researching salt sensitivity through measuring aberrant red blood cell based potassium efflux levels. In addition, the present invention provides methods for treating conditions involving salt sensitivity (e.g., hypertension), preventing the onset of conditions involving salt sensitivity, identifying individuals at risk for developing salt sensitivity and related conditions, identifying new types of treatment for salt sensitivity and related conditions, and evaluating the effectiveness of treatments for conditions involving salt sensitivity (e.g., hypertension).

### CROSS-REFERENCE TO RELATED APPLICATIONS

The present application is a Section 371 U.S. national stage entry of pending International Patent Application No. PCT/US2008/051320, International Filing Date Jan. 17, 2008, which claims the benefit of expired Provisional Patent Application No. 60/881,415, filed Jan. 19, 2007, all of which are hereby incorporated by reference in their entireties.

### STATEMENT REGARDING FEDERALLY SPONSORED RESEARCH OR DEVELOPMENT

None.

### FIELD OF THE INVENTION

The present invention provides methods for measuring, detecting, diagnosing, treating, and researching salt sensitivity and related conditions. In particular, the present invention provides methods for measuring, detecting, diagnosing, treating, and researching salt sensitivity through measuring aberrant red blood cell based potassium efflux levels. In addition, the present invention provides methods for treating conditions involving salt sensitivity (e.g., hypertension), preventing the onset of conditions involving salt sensitivity, identifying individuals at risk for developing salt sensitivity and related conditions, identifying new types of treatment for salt sensitivity and related conditions, and evaluating the effectiveness of treatments for conditions involving salt sensitivity (e.g., hypertension).

### BACKGROUND

Hypertension affects more than 65 million adult Americans, and prehypertension (blood pressure between 120/80 mmHg and 139/89 mmHg) affects millions more. Salt sensitivity, defined as a 10% increase in mean arterial blood pressure after consumption of a diet high in salt (see, e.g., Kawasaki T, et al., Am J Med 1978, 64:193-198; Weinberger MH, et al., Hypertension 2001, 37:429 432; Bihorac A, et al., Am J Hypertens 2000, 13:864-872; de Wardener HE, J Hum Hypertens 2002, 16:213-223; Hu G, et al., Curr Hypertens Rpt 2002, 4:13-17; He J, et al., JAMA 1999, 282:2027-2034; Tuomilehto J, et al., Lancet 2001, 357:848-851; Morimoto A, et al., Lancet 1997, 350:1734-1737; Sanada, H, et al., Clin. Chem. 2006, 352-360; each herein incorporated by reference in their entireties), affects 58 million Americans without hypertension and can lead to morbidity and mortality rates similar to those of hypertension.

New methods for identifying subjects suffering from who are suspected of having salt sensitivity are needed. In particular, methods for identifying subjects suffering from who are suspected of having salt sensitivity that are faster and more reliable than previous salt detection methods are needed.

### SUMMARY OF THE INVENTION

The present invention provides methods for measuring, detecting, diagnosing, treating, and researching salt sensitivity and related conditions. In particular, the present invention provides methods for measuring, detecting, diagnosing, treating, and researching salt sensitivity through measuring aberrant red blood cell based potassium efflux levels. In addition, the present invention provides methods for treating conditions involving salt sensitivity (e.g., hypertension), preventing the onset of conditions involving salt sensitivity, identifying individuals at risk for developing salt sensitivity and related conditions, identifying new types of treatment for salt sensitivity and related conditions, and evaluating the effectiveness of treatments for conditions involving salt sensitivity (e.g., hypertension).

In certain embodiments, the present invention provides a method for detecting salt sensitivity in a subject, comprising providing first and second red blood cell samples from a subject; exposing the first red blood cell sample to a salt-based agent and measuring the red blood cell based potassium efflux; exposing the second red blood cell sample to a salt-based agent and a potassium efflux inhibiting agent and measuring the red blood cell based potassium efflux; quantifying the difference in measured red blood cell based potassium efflux between the first and second samples; and detecting the presence or absence of salt sensitivity in the subject based upon the quantified difference in measured red blood cell based potassium efflux between the first and second samples.

The method is not limited to a particular salt-based agent (e.g., saline). The method is not limited to a particular potassium efflux inhibiting agent. Examples of potassium efflux inhibiting agents include, but are not limited to, potassium sparing drugs (e.g., spironolactone, eplerone, amiloride, triamterene, and any mineralocorticoid receptor blocking agent), potassium channel blockers (e.g., apamin, clotramazole, cetiedil, charybdotoxin, TEA, Ba⁺⁺), nitric oxide donors (e.g., nitroglycerin, nitroprusside, nicorandil, sydnonimines agents, statin agents, l-arginine agents, tetrahydrobiopterin) and antioxidants (e.g., polyphenolic agents, ascordbic acid, fluvastatin, selenium, α-tocopherol).

The method is not limited to a particular method for detecting. In some embodiments, the diagnosing comprises comparing the quantified difference in measured red blood cell based potassium efflux between the first and second samples with established salt sensitivity measured potassium concentration threshold levels.

In certain embodiments, the present invention provides a method of treating a subject suffering from salt sensitivity comprising: administering to the subject an agent designed to prevent red blood cell based potassium efflux. Any type of agent designed to prevent red blood cell based potassium efflux may be administered, including, but not limited to, potassium sparing drugs (e.g., spironolactone, eplerone, amiloride, triamterene, and any mineralocorticoid receptor blocking agent), potassium channel blockers (e.g., apamin, clotramazole, cetiedil, charybdotoxin, TEA, Ba⁺⁺), nitric oxide donors (e.g., nitroglycerin, nitroprusside, nicorandil, sydnonimines agents, statin agents, l-arginine agents, tetrahydrobiopterin) and antioxidants (e.g., polyphenolic agents, ascordbic acid, fluvastatin, selenium, α-tocopherol).

In certain embodiments, the present invention provides a method of evaluating the effectiveness of a salt sensitivity treatment for an individual, comprising administering the salt sensitivity treatment to the individual, obtaining red blood cell based potassium efflux levels for the individual, and evaluating the effectiveness of the salt sensitivity treatment based upon the obtained red blood cell based potassium efflux levels. In some embodiments, the method further comprises the step of obtaining a baseline red blood cell based potassium efflux level for the individual prior to the administering of the salt sensitivity treatment. In some embodiments, the obtaining red blood cell based potassium efflux levels occurs during the course of the salt sensitivity treatment. In some embodiments, the method further comprises the step of obtaining a post-treatment red blood cell based potassium efflux level. In some embodiments, the method further comprises the step of adjusting or monitoring the salt sensitivity treatment so as to maintain the red blood cell based potassium efflux levels at or above a desired red blood cell based potassium efflux level.

In some embodiments, the obtaining red blood cell based potassium efflux levels comprises a collection of a blood sample from the individual and analysis of the blood sample. In some embodiments, the analysis of the blood sample comprises measurement of the red blood cell based potassium efflux levels.

The method is not limited to a particular type or form of salt sensitivity treatment. In some embodiments, the salt sensitivity treatment comprises life-style modification. The method is not limited to a particular type or form of life-style modification. In some embodiments, the life-style modification comprises one or more life-style modifications selected from the group consisting of a dietary change, a reduction in alcohol intake, an increase in aerobic activity, a reduction or elimination of nicotine intake, an adequate intake of dietary calcium and magnesium, and a reduction of sodium intake. In some embodiments, the salt sensitivity treatment comprises a pharmacological treatment. The method is not limited to a particular type or form of pharmacological treatment. In some embodiments, the pharmacological treatment comprises one or more pharmacological treatments including, but not limited to, administration of one or more potassium sparing drugs (e.g., spironolactone, eplerone, amiloride, triamterene, and any mineralocorticoid receptor blocking agent), administration of one or more potassium channel blockers (e.g., apamin, clotramazole, cetiedil, charybdotoxin, TEA, Ba⁺⁺), administration of one or more nitric oxide donors (e.g., nitroglycerin, nitroprusside, nicorandil, sydnonimines agents, statin agents, l-arginine agents, tetrahydrobiopterin), administration of one or more antioxidants (e.g., polyphenolic agents, ascordbic acid, fluvastatin, selenium, α-tocopherol), administration of one or more aldosterone blocking drugs, administration of one or more potassium oral supplements, and administration of one or more anti-hypertensive drugs. In some embodiments, the salt sensitivity treatment is an experimental treatment. The method is not limited to a particular type or form of experimental treatment. In some embodiments, the salt sensitivity treatment is a combination of at least two of a life-style modification, a pharmacological treatment, and/or an experimental treatment.

In certain embodiments, the present invention provides a method of preventing the onset of salt sensitivity, comprising administering to an individual at risk for developing salt sensitivity a treatment configured to decrease the individual's red blood cell based potassium efflux levels below a predetermined red blood cell based potassium efflux level threshold, obtaining at least one measurement of the individual's red blood cell based potassium efflux levels during the course of the treatment, and monitoring the effectiveness of the treatment through comparison of the measured red blood cell based potassium efflux levels with the predetermined red blood cell based potassium efflux level threshold.

The method is not limited to a particular type or form of treatment configured to decrease the individual's red blood cell based potassium efflux levels below a predetermined red blood cell based potassium efflux level threshold. In some embodiments, the treatment comprises life-style modification. The method is not limited to a particular type or form of life-style modification. In some embodiments, the life-style modification comprises one or more life-style modifications selected from the group consisting of a dietary change, a reduction in alcohol intake, an increase in aerobic activity, a reduction or elimination of nicotine intake, an adequate intake of dietary calcium and magnesium, and a reduction of sodium intake. In some embodiments, the treatment comprises a pharmacological treatment. The method is not limited to a particular type or form of pharmacological treatment. In some embodiments, the pharmacological treatment comprises one or more pharmacological treatments selected from the group consisting of administration of one or more potassium sparing drugs (e.g., spironolactone, eplerone, amiloride, triamterene, and any mineralocorticoid receptor blocking agent), administration of one or more potassium channel blockers (e.g., apamin, clotramazole, cetiedil, charybdotoxin, TEA, Ba⁺⁺), administration of one or more nitric oxide donors (e.g., nitroglycerin, nitroprusside, nicorandil, sydnonimines agents, statin agents, l-arginine agents, tetrahydrobiopterin), administration of one or more antioxidants (e.g., polyphenolic agents, ascordbic acid, fluvastatin, selenium, α-tocopherol), administration of one or more aldosterone blocking drugs, administration of one or more potassium oral supplements, and administration of one or more anti-hypertensive drugs. In some embodiments, the treatment is an experimental treatment. The method is not limited to a particular type or form of experimental treatment. In some embodiments, the salt sensitivity treatment is a combination of at least two of a life-style modification, a pharmacological treatment, and/or an experimental treatment.

In some embodiments, the present invention provides drug screening assays (e.g., to screen for new drugs for treating salt sensitivity). The screening methods of the present invention utilize the methods for measuring salt sensitivity provided in the present invention. For example, in some embodiments, the present invention provides methods of screening for compounds that alter (e.g., increase or decrease), directly or indirectly, red blood cell based potassium efflux. In some embodiments, candidate compounds are antisense agents (e.g., siRNAs, oligonucleotides, etc.) directed against pathways associated with red blood cell based potassium efflux. In some embodiments, candidate compounds are evaluated for their ability to alter the amount of red blood cell based potassium efflux by contacting a candidate compound with a packed red blood cells in the presence of a salt-based agent (e.g., saline) and then measuring the potassium efflux concentration.

In some embodiments, the present invention provides kits for the measurement, diagnosis, treatment and/or study of salt sensitivity through measurement of red blood cell based potassium efflux. In some embodiments, the kits contain reagents for measuring red blood cell based potassium efflux within a blood sample, for diagnosing salt sensitivity based upon a measured potassium efflux concentration, and for treating or preventing salt sensitivity.

In certain embodiments, the present invention provides devices (e.g., hand-held devices) for measuring red blood cell related potassium efflux from a blood sample. The devices of the present invention are not limited to a particular method for measuring red blood cell related potassium efflux from a blood sample. In some embodiments, the device is configured to 1) separate a blood sample into first and second samples of red blood cells, 2) induce potassium efflux within the first sample of packed red blood cells with a salt-based agent, and induce potassium efflux in the second sample of packed red blood cells with a salt-based agent and a potassium efflux suppressing agent, and 3) measuring the difference in potassium efflux between the first and second samples of packed red blood cells. In some embodiments, the devices have therein a processor for the calculating potassium efflux differences. In some embodiments, the processor is configured to interact (e.g., wireless) with software configured to accomplish the comparing of the measured erythrocyte potassium level with the predetermined erythrocyte potassium level threshold. The devices of the present invention are not limited to a particular manner of displaying the measured red blood cell related potassium efflux (e.g., digital display).

In certain embodiments, the present invention provides kits for measuring red blood cell related potassium efflux from a blood sample. In some embodiments, the kit comprises a blood collection vessel and a device for measuring red blood cell related potassium efflux from a blood sample. In some embodiments, the device is a hand-held device. In some embodiments, the device is a desktop device. In some embodiments, the blood collection vessel with a lancet with a cellulose strip having thereon antibodies for red blood cells.

### DETAILED DESCRIPTION

Essential hypertension is a major cause of morbidity and mortality in industrialized populations of the world and one for which there is no known cause. In the United States, the prevalence of hypertension increases with age, and at about age 55, the prevalence becomes greater in women versus men (see, e.g., Burt VL, et al., 1995 Hypertension. 26:60-69; incorporated herein by reference in its entirety). More than half of white and three fourths of African-American (AA) women will develop hypertension by age 65 to 74 years.

Blood pressure of a subset of the human population rises as a result of an increase in the intake of salt (e.g., sodium chloride) (see, e.g., Weinberger, M. H., et al., 1996 Hypertension 27:481-490; incorporated herein by reference in its entirety). Individuals who manifest this trait are salt sensitive. As sodium (and chloride) balance must be preserved to sustain life, salt-sensitive and salt-resistant subjects maintain sodium balance. What distinguishes salt-sensitive from salt-resistant subjects is that in the face of a high-salt intake, salt-sensitive subjects raise their blood pressure, ultimately maintaining sodium balance by resorting to, for example, pressure natriuresis (see, e.g., Kimura G, et al., 1997 J Hypertens. 15:1055-1061; incorporated herein by reference in its entirety). However, a habitually high salt consumption in susceptible individuals may exert biological effects other than salt-evoked blood pressure elevation, including, for example, left ventricular hypertrophy, stiffness of conduit arteries, kidney disorders, end-organ damage, and stroke (see, e.g., de Wardener HE, et al., 2002 J. Hum. Hypertens. 6:213-233; Aviv A. 2001 Arch Int Med. 161:507-510; Messerli FH, et al., 1997 Arch Int Med. 57:2449-2452; each incorporated herein by reference in their entireties).

Acute blood pressure elevation with increasing salt intake (salt sensitivity) is commonly reported in large segments of the population, especially in those with renal disease, diabetes, obesity, hypertension, and older age (see, e.g., Williams GH, et al., 1987 Am J Kidney Dis. 10 (suppl 1): 39-44; Weinberger MH, 1996 Hypertension. 27: 481-490; each herein incorporated by reference in their entireties). Blood pressure sensitivity to salt might also predict chronic blood pressure elevation as normotensives with this trait have an increased risk for developing hypertension (see, e.g., Williams GH, et al., 1987 Am J Kidney Dis. 10 (suppl 1): 39-44; Svetkey LP, et al., 1997 Hypertension 29:918-922; each herein incorporated by reference in their entireties).

A variety of genetic and environmental factors modulate the effects of dietary sodium on cardiovascular function (e.g., blood pressure), including, but not limited to, diet quality, age, body mass and race/ethnicity as well as the role of K depletion. For example, salt sensitivity in post-menopausal women is correlated with diminished ovarian hormone expression (see, e.g., Tominaga, et al., 1991 J Hum Hypertens. 5: 495-500; Schulman, et al., 2006 Hypertens. 47(6): 1168-1174; each herein incorporated by reference in their entireties). End-organ damage (e.g., kidney failure, heart failure, proteinuria, retinopathy) is correlated with salt sensitivity (see, e.g., Maitland, et al., 2006 Circulation 114(9):905-911; incorporated herein by reference in its entirety). In addition, salt sensitivity is associated with variations in the GRK4 gene (e.g., R65L, A142V, and A486V) (see, e.g., Sanada, H., et al., 2006 Clinical Chemistry 52(3):352-360; incorporated herein by reference in its entirety).

However, the pathophysiology of salt sensitivity and its progression to hypertension is poorly understood. This is further complicated by the significant heterogeneity in methods of defining salt sensitivity (see, e.g., Falkner B. 1988 J Am Coll Nutr. 1988; 7: 35-41; Sekihara H, et al., 1979 J Clin Endocrinol Metab. 48: 143-147; Sullivan J M, et al., 1988 Hypertension 11:717-723; Skrabal F, et al., 1984 Hypertension. 6: 152-158; Bose D, et al., 1988 Br J Pharmacol. 93: 453-461; each herein incorporated by reference in their entireties). The definition of salt sensitivity has generally been based on the differences between BP after a low sodium intake, such as 9 mmol/day (ca. 0.5 g of NaCl), and that after a high sodium intake, such as 249 mmol/day (see, e.g., Kawasaki T, et al., 1978 Am J Med. 64:193-198; incorporated herein by reference in its entirety). A difference of at least 10% between the low- and high-salt mean arterial pressures is typically regarded as indicating salt-sensitivity, and a smaller difference as identifying a non-salt sensitive subject, after salt loading (see, e.g., Kawasaki T, et al., 1978 Am J Med. 64:193-198; incorporated herein by reference in its entirety). This technique, however, typically takes about two weeks to obtain a salt sensitivity measurement, and has limited reproducibility. Another technique for defining salt sensitivity involves measurement of BP after an intravenous infusion of 2 liters of normal (0.9%) saline (308 mmol or 18 g of NaCl) for 4 h, and on the next day, measurement of BP again after sodium and volume depletion induced by a low sodium diet (10 mmol) and furosemide administration, in 378 healthy volunteers and 198 subjects with essential hypertension (see, e.g., Weinberger M, et al., 1986 Hypertension 8(6 Pt2) Suppl II:127-134; incorporated herein by reference in its entirety). Those in whom mean arterial BP decreased by at least 10 mmHg after sodium and volume depletion were considered sodium-sensitive, and those with a decrease of 5 mmHg or less (including an increase in pressure) were considered sodium-resistant. Subjects with a decrease in BP between 6 and 9 mmHg were classified as indeterminate. This technique is sub-optimal as infusion of isotonic saline and diuretic medication is intrusive. As such, non-invasive and faster methods for measuring salt sensitivity are needed.

The present invention provides improved methods for measuring, detecting, diagnosing, treating, and researching salt sensitivity through measurement of aberrant red blood cell based potassium efflux levels. Potassium (K⁺) in vascular smooth muscle cells (VSMC) and in the endothelium is a very important factor in vasodilatation. In VSMC, K⁺ efflux through Ca²⁺-activated K⁺ channels (K_{Ca}) causes membrane potential hyperpolarization and closes voltage-dependent Ca²⁺ channels, which leads to vasodilatation (see, e.g., Jaggar, J. H., et al., 1998 Acta Physiol Scand 164:577-587; herein incorporated by reference in its entirety). In the endothelium, K⁺ efflux, through intermediate conductance K_{Ca} (IK_{Ca}), hyperpolarize VSMC via electrical coupling between endothelium and VSMC (see, e.g., Sandow, S. L. & Hill, C. E., 2000, Cir Res 86:341-346; herein incorporated by reference in its entirety), producing hyperpolarization and closing voltage-dependent Ca²⁺ channels. Stimulation of endothelial cells results in an outwardly rectifying K⁺ current (see, e.g., Coleman, H. A., et al., 2001, J. Physiol. 531:359-373; herein incorporated by reference in its entirety) and the combination of the K⁺ channel inhibitors apamin and charybdotoxin completely prevents the hyperpolarizing and vasodilating action of endothelium-derived hyperpolarizing factor (EDHF) in the rat hepatic artery (see, e.g., Anderson, A. J., et al., Br. J. Pharmacol. 95:1329-1335; herein incorporated by reference in its entirety). This observation indicates that K_{Ca} with the pharmacological characteristics of small and intermediate conductance K_{Ca} are involved in the EDHF vasodilatation.

Red blood cell (RBC) based potassium efflux is also affected by IK_{Ca} (see, e.g., Del Carlo, B., et al., 2002, Biochim Biophys Acta, 558:133-141; Brugnara C, et al., 1993, J Biol Chem 268: 8760-8768; each herein incorporated by reference in their entireties). Ciclazindol, which abolishes EDHF relaxation in the presence of apamin, inhibits K_{Ca} in red blood cells (see, e.g., Anderson, A. J., et al., Br. J. Pharmacol. 95:1329-1335; herein incorporated by reference in its entirety) and cromakalim, a known K⁺ channel activator with vasodilator properties, opens K_{Ca} in RBC (see, e.g., Lijnen, P., et al., 1989, J. Hypertens. 7:403-407; herein incorporated by reference in its entirety). In addition, increased K⁺ efflux occurs in RBC treated with 8Br-cGMP, for example, by activation of I K_{Ca} as it occurs in VSMC (see, e.g., Price, J. M., et al., 1997, Life Sci. 61:1185-1192; herein incorporated by reference in its entirety). As such, K⁺ changes occurring in RBC are reflective of K⁺ changes in the VSMC or in the endothelium (see, e.g., Delgado, M. C. & Delgado-Almeida, A., 2003, J. Human Hypertens. 17:313-318; herein incorporated by reference in its entirety).

Hypertensive individuals and their offspring have been shown to have lower intracellular potassium (e.g., effluxed potassium) than normotensive controls, and hypertensive individuals and their offspring have been shown to have increased intermediate conductance calcium-activated potassium channel activity (see, e.g., Delgado, et al., 2003 J. Hum. Hypertens. 17(5):313-318; incorporated herein by reference in its entirety). Chronic dietary potassium restriction has been shown to increase the BP in young rats and induce salt sensitivity involving increased renin-angiotensin activity and induction of tubulointerstitial injury (see, e.g., Ray, et al., 2001 Kidney Intl. 59(5):1850-1858; incorporated herein by reference in its entirety).

The present invention provides methods whereby measurement of red blood cell related potassium efflux represents a non-invasive and fast method for measuring salt sensitivity.

The present invention is not limited to a particular manner of measuring red blood cell related potassium efflux for purposes of measuring salt sensitivity. In some embodiments, the present invention provides in vitro methods for measuring red blood cell related potassium efflux. The present invention is not limited to a particular type of in vitro method for measuring red blood cell related potassium efflux. In some embodiments, the in vitro method involves, for example, measuring red blood cell related potassium efflux from a blood sample obtained from a subject. As used herein, the term “subject,” “individual,” or “patient” refers to any and all kinds or type of organisms. Such organisms preferably include, but are not limited to, mammals (e.g., murines, simians, equines, bovines, porcines, canines, felines, and the like), and most preferably includes humans. The term “individual” is not limited to a particular gender or age. The methods are not limited to a particular type or amount of blood sample. In some embodiments, the blood sample is a venous blood sample obtained through, for example, standard venipuncture techniques. In some embodiments, a blood collection vessel is used to collect an individual's blood. The present invention is not limited to a particular type or kind of blood collection vessel. In some embodiments, the blood collection vessel is a vacuum tube (e.g., a heparinized vacuum tube). In some embodiments, the blood collection vessel is a lancet configured to collect blood from an extremity (e.g., a finger tip). In some embodiments, the blood collection vessel is a cellulose strip from which the blood, for example, obtained with a lancet is supplied to the cellulose strip. In some embodiments, the blood collection vessel is a standard syringe of any desired size. In preferred embodiments, the blood collection vessel is configured to obtain a blood sample from an individual for purposes of measuring red blood cell related potassium efflux levels within the blood sample.

The present invention is not limited to a particular method of measuring red blood cell related potassium efflux from a blood sample. In some embodiments, the method comprises 1) separating the blood sample into first and second samples of packed red blood cells, 2) inducing potassium efflux within the first sample of packed red blood cells with a salt-based agent, and inducing potassium efflux in the second sample of packed red blood cells with a salt-based agent and a potassium efflux suppressing agent, and 3) measuring the difference in potassium efflux between the first and second samples of packed red blood cells.

The present invention is not limited to a particular manner of separating a blood sample into first and second samples of packed red blood cells. In some embodiments, the plasma is removed from the blood sample through, for example, centrifugation, and the remaining packed red blood cells separated into first and second samples of packed red blood cells. The first and second samples of packed red blood cells are not limited to a particular quantity size. In some embodiments, the quantity of packed red blood cells within the first and second samples is sufficient to obtain potassium efflux measurements (e.g., 100 μliters per sample). In some embodiments, the first and second samples are contained within separate vials.

The present invention is not limited to a particular method of inducing potassium efflux in the first sample of packed red blood cells with a salt-based agent. In some embodiments, the first sample of packed red blood cells is exposed to a salt-based agent (e.g., saline) so as to induce potassium efflux. In some embodiments, the first sample of packed red blood cells is exposed to a salt-based agent (e.g., saline) so as to induce potassium efflux within one hour of obtaining the blood sample. In some embodiments, the first sample of packed red blood cells is exposed to a salt-based agent (e.g., saline) so as to induce potassium efflux for a period of, for example, one hour within, for example, a water bath heated to, for example, 37° C.

The present invention is not limited to a particular method of inducing potassium efflux in the second sample of packed red blood cells with a salt-based agent and a potassium efflux suppressing agent. In some embodiments, the second sample is exposed (e.g., simultaneously exposed) to a salt-based agent (e.g., saline) and a potassium efflux suppressing agent. Examples of potassium efflux suppressing agents include, but are not limited to, potassium sparing drugs (e.g., spironolactone, eplerone, amiloride, triamterene, and any mineralocorticoid receptor blocking agent), potassium channel blockers (e.g., apamin, clotramazole, cetiedil, charybdotoxin, TEA, Ba⁺⁺), nitric oxide donors (e.g., nitroglycerin, nitroprusside, nicorandil, sydnonimines agents, statin agents, l-arginine agents, tetrahydrobiopterin) and antioxidants (e.g., polyphenolic agents, ascordbic acid, fluvastatin, selenium, α-tocopherol). In some embodiments, the second sample is exposed (e.g., simultaneously exposed) to a salt-based agent (e.g., saline) and a potassium efflux suppressing agent (e.g., spironolactone, eplerone, amiloride, triamterene, and any mineralocorticoid receptor blocking agent), within one hour of obtaining the blood sample. In some embodiments, the second sample is exposed (e.g., simultaneously exposed) to a salt-based agent (e.g., saline) and a potassium efflux suppressing agent (e.g., spironolactone, eplerone, amiloride, triamterene, and any mineralocorticoid receptor blocking agent) for a period of, for example, one hour within, for example, a water bath heated to, for example, 37° C. The present invention is not limited to a particular type, kind or amount of salt-based agent (e.g., 5 ml normal saline solution-0.9% w/v of NaCl). The present invention is not limited to a particular type, kind or amount of potassium efflux suppressing agent (e.g., spironolactone, eplerone, amiloride, triamterene, and any mineralocorticoid receptor blocking agent). In some embodiments, both the first and second samples of packed red blood cells are exposed to the same type and amount of salt-based agent.

The present invention is not limited to a particular method for measuring differences in potassium efflux between first and second samples of packed red blood cells. In some embodiments, the effluxed material from the first and second samples is separated from the red blood cells through standard laboratory techniques (e.g., via centrifugation). The present invention is not limited to a particular method of separating the effluxed material from the first and second samples. In some embodiments, the potassium concentration within the separated effluxed material is measured. The present invention is not limited to a particular manner of measuring the potassium concentration of the effluxed material (e.g., through standard laboratory techniques). In some embodiments, the difference in measured red blood cell potassium efflux concentrations between 1) a red blood cell sample exposed only to a salt-based agent and 2) a sample exposed to both a salt-based agent and a potassium efflux suppressing agent is calculated. In some embodiments, the calculated difference in measured red blood cell potassium efflux concentrations between 1) a red blood cell sample exposed only to a salt-based agent and 2) a sample exposed to both a salt-based agent and a potassium efflux suppressing agent is referred to as the red blood cell based potassium efflux delta. In some embodiments, the calculated difference in measured red blood cell potassium efflux concentrations between 1) a red blood cell sample exposed only to a salt-based agent and 2) a sample exposed to both a salt-based agent and a potassium efflux suppressing agent may be used to diagnose a subject as having or not having salt sensitivity, and/or as being at risk or not at risk for developing salt sensitivity.

The present invention is not limited to a particular method of detecting or diagnosing salt sensitivity or related conditions in a subject, or risk of developing salt sensitivity or related conditions in a subject. In some embodiments, diagnosis of salt sensitivity in a subject is accomplished through comparing a subject's red blood cell based potassium efflux delta to established thresholds. For example, in some embodiments, a subject's red blood cell based potassium efflux delta is compared with established red blood cell based potassium efflux delta threshold levels (e.g., established red blood cell based potassium efflux delta threshold levels for low risk for developing salt sensitivity; established red blood cell based potassium efflux delta threshold levels for medium risk for developing salt sensitivity; established red blood cell based potassium efflux delta threshold levels for high risk for developing salt sensitivity; established red blood cell based potassium efflux delta threshold levels for having salt sensitivity versus not having salt sensitivity; established red blood cell based potassium efflux delta threshold levels for salt sensitivity; established red blood cell based potassium efflux delta threshold levels for not having salt sensitivity). Established red blood cell based potassium efflux delta threshold levels may be generated from any number of sources, including but not limited to, groups of subjects having salt sensitivity, groups of subjects not having salt sensitivity, groups of subjects having hypertension and salt sensitivity, groups of subjects not having hypertension or salt sensitivity, groups of subjects having hypertension but not having salt sensitivity, groups of subjects not having hypertension but having salt sensitivity, groups of subjects having salt sensitivity and a particular form of treatment, etc. Established red blood cell based potassium efflux delta threshold levels may also be gender based, age based, and condition based (e.g., hypertension based). Any number of subjects within a group may be used to generate an established red blood cell based potassium efflux delta threshold levels (e.g., 5 subjects, 10 subjects, 20, 30, 50, 500, 5000, 10,000, etc.). Established red blood cell based potassium efflux delta threshold levels may be generated with any type or source of bodily sample from a subject (e.g., including but not limited to, red blood cells, plasma, serum, whole blood, mucus, and urine).

In some embodiments, the present invention provides devices for measuring red blood cell related potassium efflux from a blood sample and detecting salt sensitivity. The device is not limited to a particular size. In some embodiments, the device is a handheld device. In some embodiments, the device is able to fit within a user's pocket (e.g., pants pocket, laboratory coat pocket). In some embodiments, the weight of the hand-held device is less than 5 pounds. In some embodiments, the devices are configured for use with a health care professional (e.g., a physician, a nurse). In some embodiments, the devices are configured for use with a non-health care professional.

The devices of the present invention are not limited to a particular method for measuring red blood cell related potassium efflux from a blood sample. In some embodiments, the device is configured to 1) separate a blood sample into first and second samples of red blood cells, 2) induce potassium efflux within the first sample of packed red blood cells with a salt-based agent, and induce potassium efflux in the second sample of packed red blood cells with a salt-based agent and a potassium efflux suppressing agent, and 3) measuring the difference in potassium efflux between the first and second samples of packed red blood cells.

The devices of the present invention are not limited to a particular manner of separating a blood sample into first and second red blood cell samples. In some embodiments, the devices are designed to accept cellulose strips (or other solid surfaces) having antibodies (e.g., a predefined number of antibodies so as to bind a known amount of sample) specific for red blood cells. In some embodiments, the devices have a microcentrifuge for separating red blood cells from blood samples.

The devices of the present invention are not limited to a particular manner of inducing potassium efflux with the red blood cell samples. In some embodiments, the devices are configured to induce potassium efflux within the first sample of red blood cells with a salt-based agent, and induce potassium efflux in the second sample of packed red blood cells with a salt-based agent and a potassium efflux suppressing agent. In some embodiments, the devices are configured to induce potassium efflux for each sample of red blood cells under “high salt” conditions, “low salt” conditions, and “blocking agent” conditions.

The devices of the present invention are not limited to a particular manner of measuring the differences in potassium efflux between the first and second samples of packed red blood cells. In some embodiments, the devices are designed to measure such differences with imaging agents (e.g., bioluminescence, fluorescence, etc.). In some embodiments, the devices are configured to utilize flame emission spectroscopy for purposes of measuring the difference in potassium efflux levels. In some embodiments, the device is configured to utilize potassium selective electrodes for purposes of measuring the individual's erythrocyte potassium level.

In some embodiments, the devices have therein a processor for the calculating potassium efflux differences and detecting salt sensitivity. In some embodiments, the processor is configured to interact (e.g., wireless) with software configured to accomplish the comparing of the measured erythrocyte potassium level with the predetermined erythrocyte potassium level threshold. In some embodiments, the devices are configured to interact (e.g., wireless) with a database containing information for a patient (e.g., a hospital database). In some embodiments, the devices have a memory for storing measured red blood cell related potassium efflux over a period of time. In some embodiments, the memory is at least 100 Mb (e.g., 150 Mb, 700 Mb, 1 gig, 100 gigs, 1 terabyte, 100 terabytes). In some embodiments, the interacting is wireless communication to the Internet.

The devices of the present invention are not limited to a particular manner of displaying the measured red blood cell related potassium efflux and the presence or absence of salt sensitivity. In some embodiments, the devices have an audible system for presenting results. In some embodiments, the results are presented in a digital display. In some embodiments, the actual measured red blood cell related potassium efflux is presented (e.g., for use with a health care professional). In some embodiments, the results are presented in a “yes” or “no” format indicating whether or not the blood sample is experiencing salt sensitivity. In some embodiments, the results are presented as a recommendation to seek health care professional assistance. In some embodiments, the devices are configured to display a user's risk for salt sensitivity. In some embodiments, the devices are configured to display a numerical based salt sensitivity value.

In some embodiments, the present invention provides kits for measuring red blood cell related potassium efflux from a blood sample. The present invention is not limited to particular parts of the kit. In some embodiments, the kit comprises a blood collection vessel and a device for measuring red blood cell related potassium efflux from a blood sample. In some embodiments, the device is a hand-held device. In some embodiments, the device is a desktop device. In some embodiments, the blood collection vessel with a lancet with a cellulose strip having thereon antibodies for red blood cells.

In some embodiments, the present invention provides home test kits for individual use. In some embodiments, the home test kit comprises a hand-held device for measuring red blood cell related potassium efflux from a blood sample and instructions for operating the hand-held device. In some embodiments, the home test kit comprises test strips for measuring an erythrocyte potassium level from a bodily sample (e.g., blood sample) and instructions for operating the test strips. In some embodiments, the test strips and hand-held device are configured to display a user's risk for salt sensitivity. In some embodiments, the test strips and hand-held device are configured to display a numerical based salt sensitivity value. In some embodiments, the home test kits comprise instructions for interpreting displayed results.

The present invention provides methods for treating or preventing salt sensitivity and related conditions. The present invention is not limited to a particular method for treating or preventing salt sensitivity and/or complications associated with salt sensitivity (e.g., hypertension, end-organ damage, stroke, renal failure, proteinuria, retinopathy). In some embodiments, the present invention provides methods for treating or preventing salt sensitivity through preventing (e.g., reducing, inhibiting) red blood cell based potassium efflux (e.g., a low sodium diet, fitness improvement, administration of supplemental potassium, preserving angiotensin type I activity). The present invention is not limited to a particular method for preventing red blood cell based potassium efflux. The present invention is not limited to a particular type of treatment or combination of treatments (e.g., preventing the onset of salt sensitivity in individuals identified as being at high or low risk for developing salt sensitivity; treating individuals diagnosed as having salt sensitivity; experimental treatment). In some embodiments, the treatment includes a life-style change, an experimental treatment, and/or a pharmacological treatment.

The present invention is not limited to a particular type of life-style change. In some embodiments, the life-style change comprises a dietary change (e.g., reduced intake of dietary saturated fat and cholesterol). In some embodiments, the life-style change involves a reduction in alcohol intake (e.g., limiting alcohol intake to no more than 1 oz (30 mL) of ethanol (e.g., 24 oz (720 mL) of beer, 10 oz (300 mL) of wine, 2 oz (60 mL) of 100-proof whiskey) per day or 0.5 (15 mL) ethanol per day for women and people of lighter weight). In some embodiments, the life-style change involves an increase in aerobic activity for the individual (e.g., increasing aerobic activity to 30-45 minutes most days of the week). In some embodiments, the life-style change involves reduction or elimination of nicotine intake (e.g., stopping smoking). In some embodiments, the life-style change involves adequate intake of dietary calcium and magnesium. In some embodiments, the life-style change involves reduction of sodium intake to no more than 100 mmol/d (2.4 g sodium or 6 g sodium chloride). In some embodiments, the life-style change comprises a combination of life-style changes.

The present invention is not limited to a particular type of pharmacological treatment. In some embodiments, the pharmacological treatment comprises one or more pharmacological treatments comprising administration of one or more drugs including, but not limited to, hydrochlorothiazide, spironolactone, eplerone, any mineralocorticoid receptor blocking agent, amiloride, furosemide, prazosin, atenolol, metoprolol, propranolol, labetalol, carvedilol, hydralazine, minoxidil, diltiazem, verapamil, nifedipine, captopril, enalapril, lisinopril, ramipril, losartan, valsartan, eprosartan, olmesartan, eplerenone, methyldopa, clonidine. In some embodiments, the pharmacological treatment comprises one or more pharmacological treatments selected from the group consisting of administration of one or more potassium sparing drugs (e.g., spironolactone, eplerone, amiloride, triamterene, and any mineralocorticoid receptor blocking agent), administration of one or more potassium channel blockers (e.g., apamin, clotramazole, cetiedil, charybdotoxin, TEA, Ba⁺⁺), administration of one or more nitric oxide donors (e.g., nitroglycerin, nitroprusside, nicorandil, sydnonimines agents, statin agents, l-arginine agents, tetrahydrobiopterin), administration of one or more antioxidants (e.g., polyphenolic agents, ascordbic acid, fluvastatin, selenium, α-tocopherol), administration of one or more aldosterone blocking drugs (e.g., spironolactone, eplerone, amiloride, triamterene, and any mineralocorticoid receptor blocking agent), administration of one or more potassium oral supplements, and administration of one or more anti-hypertensive drugs (e.g., a thiazide diuretic, a loop diuretic, a beta blocker, a beta blocker with intrinsic sympathomimetic activity, a combined alpha and beta blocker, an angiotensin converting enzyme inhibitor, an angiotensin II antagonist, a calcium channel blocker, an alpha-1 blocker, a central alpha-2 agonist, or a direct vasodilator). In some embodiments, the potassium oral supplement is any form of potassium chloride (e.g., oral capsule extended release, oral powder for solution, oral powder for suspension—extended release, oral solution, oral tablet, oral tablet—extended release, and sublingual tablet). In some embodiments, the pharmacological treatment comprises administration of a combination of drugs.

The present invention is not limited to a particular type of experimental treatment. In some embodiments, the experimental treatment comprises administration of a new pharmacological agent (e.g., a new medication). In some embodiments, the experimental treatment comprises an experimental procedure involving stem cells (e.g., stem cell therapy). In some embodiments, the experimental treatment comprises an experimental procedure involving cellular therapy (e.g., therapies involving mononuclear transformed cells). In some embodiments, the experimental treatment comprises an experimental procedure involving gene therapy (e.g., gene therapy directed to correct a defect in an individual's erythrocyte potassium level pathways and related pathways (e.g., erythrocyte transport pathways)). In some embodiments, the experimental treatment comprises an experimental surgical procedure. This aspect of the present invention permits the screening of new pharmacological agents, new cellular therapies, new forms of gene therapy, new surgical procedures, new life-style modifications, and other new forms of treatment, and combinations thereof, for an ability to modify an individual's erythrocyte potassium levels.

In some embodiments, the present invention provides drug screening assays (e.g., to screen for new drugs for treating salt sensitivity). The screening methods of the present invention utilize the methods for measuring salt sensitivity provided in the present invention. For example, in some embodiments, the present invention provides methods of screening for compounds that alter (e.g., increase or decrease), directly or indirectly, red blood cell based potassium efflux. In some embodiments, candidate compounds are antisense agents (e.g., siRNAs, oligonucleotides, etc.) directed against pathways associated with red blood cell based potassium efflux. In some embodiments, candidate compounds are evaluated for their ability to alter the amount of red blood cell based potassium efflux by contacting a candidate compound with a packed red blood cells in the presence of a salt-based agent (e.g., saline) and then measuring the potassium efflux concentration.

In some embodiments, the present invention provides kits for the measurement, detection, diagnosis, treatment and/or study of salt sensitivity through measurement of red blood cell based potassium efflux. In some embodiments, the kits contain reagents for measuring red blood cell based potassium efflux within a blood sample, for diagnosing salt sensitivity based upon a measured potassium efflux concentration, and for treating or preventing salt sensitivity. The kits may comprise one or more components useful for, necessary for, or sufficient for carrying out the methods described herein.

In some embodiments, the present invention provides a method of monitoring the effectiveness of a particular treatment or combination of treatments. In some embodiments, the method of monitoring comprises evaluating the effectiveness of a treatment through monitoring the change in red blood cell based potassium efflux levels for an individual over a period of time (e.g., the course of the treatment) or maintaining red blood cell based potassium efflux levels above a desired threshold over the course of treatment. The method of monitoring is not limited to a particular series of steps. In some embodiments, the method of monitoring comprises measurement of an individual's red blood cell based potassium efflux level, administration of a treatment, measuring the individual's red blood cell based potassium efflux levels during the course of the treatment, and evaluating the effectiveness of the treatment based on the individual's red blood cell based potassium efflux levels during the course of the treatment. In some embodiments, the first measurement of the individual's red blood cell based potassium efflux level occurs after the onset of treatment. In some embodiments, the method of monitoring further comprises the step of changing a treatment (e.g., continuing a treatment, stopping a treatment, starting a new treatment, increasing a treatment, decreasing a treatment, etc.) based upon the evaluated effectiveness of an administered treatment. In some embodiments, the monitoring of the individual's red blood cell based potassium efflux levels is conducted at a plurality of time points (e.g., weekly, daily, hourly, continuously).

In some embodiments, a treatment is considered effective when an individual's red blood cell based potassium efflux levels decrease over the course of the treatment or the red blood cell based potassium efflux levels are maintained at a desired level over the course of the treatment. In some embodiments, a treatment is considered ineffective when an individual's red blood cell based potassium efflux levels increase over the course of a treatment or the red blood cell based potassium efflux levels are maintained at an undesired level over the course of the treatment.

In some embodiments, the effectiveness of a treatment is monitored in comparison to an identified red blood cell based potassium efflux level threshold. In such embodiments, a treatment is considered effective when over the course of the treatment an individual's red blood cell based potassium efflux levels remain at or below the red blood cell based potassium efflux level threshold. In some embodiments, a treatment is considered ineffective when over the course of the treatment an individual's red blood cell based potassium efflux levels remain above the red blood cell based potassium efflux level threshold.

In some embodiments, in addition to monitoring an individual's red blood cell based potassium efflux levels over a period of time, the method of monitoring involves additional steps directed toward evaluating the effectiveness of a treatment. The present invention is not limited to particular steps directed toward evaluating the effectiveness of a treatment. In some embodiments, the additional steps include, but are not limited to, analysis of CBC count, analysis of serum electrolytes, analysis of serum creatinine, analysis of serum glucose, analysis of uric acid, urinalysis, a lipid profile analysis (e.g., total cholesterol, low-density lipoprotein (LDL) and high-density lipoprotein (HDL), and triglycerides), imaging tests (e.g., electrocardiography), electrocardiograms, obtaining total body potassium measurements by bio-impedance, analysis of 12-hour urinary potassium excretion, obtaining serial plasma glucose and insulin levels, and ambulatory blood pressure monitoring.

All publications and patents mentioned in the above specification are herein incorporated by reference. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method for detecting salt sensitivity in a subject, comprising:
a) providing first and second red blood cell samples from a subject;
b) exposing said first red blood cell sample to a salt-based agent and measuring the red blood cell based potassium efflux;
c) exposing said second red blood cell sample to a salt-based agent and a potassium efflux inhibiting agent and measuring the red blood cell based potassium efflux;
d) quantifying the difference in measured red blood cell based potassium efflux between said first and second samples; and
e) detecting the presence or absence of salt sensitivity in said subject based upon said quantified difference in measured red blood cell based potassium efflux between said first and second samples.

2. The method of claim 1, wherein said salt-based agent is saline.

3. The method of claim 1, wherein said potassium efflux inhibiting agent is selected from the group consisting of spironolactone, eplerone, amiloride, and triamterene.

4. The method of claim 1, wherein said diagnosing comprises comparing said quantified difference in measured red blood cell based potassium efflux between said first and second samples with established salt sensitivity measured potassium concentration threshold levels.

5. A method of treating a subject suffering from salt sensitivity, comprising:
a) measuring red blood cell based potassium efflux in said subject with the method of claim 1,
b) administering to said subject one or more agents designed to prevent red blood cell based potassium efflux.

6. The method of claim 5, wherein said one or more agents designed to prevent red blood cell based potassium efflux is selected from the group consisting of spironolactone, eplerone, amiloride, triamterene, hydrochlorothiazide, furosemide, prazosin, atenolol, metoprolol, propranolol, labetalol, carvedilol, hydralazine, minoxidil, diltiazem, verapamil, nifedipine, captopril, enalapril, lisinopril, ramipril, losartan, valsartan, eprosartan, olmesartan, eplerenone, methyldopa, clonidine, triamterene, apamin, clotramazole, cetiedil, charybdotoxin, TEA, Ba⁺⁺, nitroglycerin, nitroprusside, nicorandil, sydnonimines agents, statin agents, l-arginine agents, tetrahydrobiopterin, polyphenolic agents, ascordbic acid, fluvastatin, selenium, α-tocopherol, eplerenone, a thiazide diuretic, a loop diuretic, a beta blocker, a beta blocker with intrinsic sympathomimetic activity, a combined alpha and beta blocker, an angiotensin converting enzyme inhibitor, an angiotensin II antagonist, a calcium channel blocker, an alpha-1 blocker, a central alpha-2 agonist, a direct vasodilator, and a potassium oral supplement.

7. A method of evaluating the effectiveness of a salt sensitivity treatment for an individual, comprising:
a) administering said salt sensitivity treatment to said individual,
b) obtaining red blood cell based potassium efflux levels for said individual, and
c) evaluating the effectiveness of said salt sensitivity treatment based upon said obtained red blood cell based potassium efflux levels.

8. The method of claim 7, further comprising the step of obtaining a baseline red blood cell based potassium efflux level for said individual prior to said administering of said salt sensitivity treatment.

9. The method of claim 7, wherein said obtaining red blood cell based potassium efflux levels occurs during the course of said salt sensitivity treatment.

10. The method of claim 7, further comprising the step of obtaining a post-treatment red blood cell based potassium efflux level.

11. The method of claim 7, further comprising the step of adjusting or monitoring said salt sensitivity treatment so as to maintain said red blood cell based potassium efflux levels at or above a desired red blood cell based potassium efflux level.

12. The method of claim 7, wherein said obtaining red blood cell based potassium efflux levels comprises a collection of a blood sample from said individual and analysis of said blood sample.

13. The method of claim 12, wherein said analysis of said blood sample comprises measurement of said red blood cell based potassium efflux levels.

14. The method of claim 7, wherein said salt sensitivity treatment comprises life-style modification.

15. The method of claim 7, wherein said salt sensitivity treatment comprises a pharmacological treatment.

16. The method of claim 7, wherein said salt sensitivity treatment comprises an experimental treatment.

17. The method of claim 14, wherein said life-style modification comprises one or more life-style modifications selected from the group consisting of a dietary change, a reduction in alcohol intake, an increase in aerobic activity, a reduction or elimination of nicotine intake, an adequate intake of dietary calcium and magnesium, and a reduction of sodium intake.

18. The method of claim 15, wherein said pharmacological treatment comprises one or more pharmacological treatments selected from the group consisting of spironolactone, amiloride, eplerone, triamterene, hydrochlorothiazide, furosemide, prazosin, atenolol, metoprolol, propranolol, labetalol, carvedilol, hydralazine, minoxidil, diltiazem, verapamil, nifedipine, captopril, enalapril, lisinopril, ramipril, losartan, valsartan, eprosartan, olmesartan, eplerenone, methyldopa, clonidine, triamterene, apamin, clotramazole, cetiedil, charybdotoxin, TEA, Ba⁺⁺, nitroglycerin, nitroprusside, nicorandil, sydnonimines agents, statin agents, l-arginine agents, tetrahydrobiopterin, polyphenolic agents, ascordbic acid, fluvastatin, selenium, α-tocopherol, eplerenone, a thiazide diuretic, a loop diuretic, a beta blocker, a beta blocker with intrinsic sympathomimetic activity, a combined alpha and beta blocker, an angiotensin converting enzyme inhibitor, an angiotensin II antagonist, a calcium channel blocker, an alpha-1 blocker, a central alpha-2 agonist, a direct vasodilator, and a potassium oral supplement.

19. A method of preventing the onset of salt sensitivity, comprising
administering to an individual at risk for developing salt sensitivity a treatment configured to decrease said individual's red blood cell based potassium efflux levels below a predetermined red blood cell based potassium efflux level threshold,
obtaining at least one measurement of said individual's red blood cell based potassium efflux levels during the course of said treatment, and
monitoring the effectiveness of said treatment through comparison of said measured red blood cell based potassium efflux levels with said predetermined red blood cell based potassium efflux level threshold.

20. The method of claim 19, wherein said treatment comprises a life-style modification.

21. The method of claim 19, wherein said treatment comprises a pharmacological treatment.

22. The method of claim 20, wherein said life-style modification comprises one or more life-style modifications selected from the group consisting of a dietary change, a reduction in alcohol intake, an increase in aerobic activity, a reduction or elimination of nicotine intake, an adequate intake of dietary calcium and magnesium, and a reduction of sodium intake.

23. The method of claim 21, wherein said pharmacological treatment comprises one or more pharmacological treatments selected from the group consisting of spironolactone, eplereone, amiloride, triamterene, hydrochlorothiazide, furosemide, prazosin, atenolol, metoprolol, propranolol, labetalol, carvedilol, hydralazine, minoxidil, diltiazem, verapamil, nifedipine, captopril, enalapril, lisinopril, ramipril, losartan, valsartan, eprosartan, olmesartan, eplerenone, methyldopa, clonidine, triamterene, apamin, clotramazole, cetiedil, charybdotoxin, TEA, Ba⁺⁺, nitroglycerin, nitroprusside, nicorandil, sydnonimines agents, statin agents, l-arginine agents, tetrahydrobiopterin, polyphenolic agents, ascordbic acid, fluvastatin, selenium, α-tocopherol, eplerenone, a thiazide diuretic, a loop diuretic, a beta blocker, a beta blocker with intrinsic sympathomimetic activity, a combined alpha and beta blocker, an angiotensin converting enzyme inhibitor, an angiotensin II antagonist, a calcium channel blocker, an alpha-1 blocker, a central alpha-2 agonist, a direct vasodilator, and a potassium oral supplement.
